(19) United States
(12) Patent Application Publication
(10) Pub. No.: US 2003/0165843 A1
(43) Pub. Date: Sep. 4, 2003

(54) Oligonucleotide library for detecting RNA transcripts and splice variants that populate a transcriptome

(76) Inventors: Avi Shoshan, New York, NY (US); Alon Wasserman, New York, NY (US); Eli Mintz, Kendall Park, NJ (US); Liat Mintz, Kendall Park, NJ (US); Simchon Faigler, Edison, NJ (US)

Correspondence Address: Sol Sheinbein, G E EHRLICH (1995) LTD, C/O Anthony Castorina, 2001 Jefferson Davis Highway Suite 207, Arlington, VA, 22202, US

(21) Appl. No.: 09/908,975

(22) Filed: Jul. 20, 2001

Related U.S. Application Data
(60) Provisional application No. 60/287,724, filed on May 2, 2001.
(60) Provisional application No. 60/221,607, filed on Jul. 28, 2000.

(51) Int. Cl. C12Q 1/68; C07H 21/04

(52) U.S. Cl. 435/6; 536/23.1; 536/24.3

(57) ABSTRACT

The present invention provides oligonucleotide libraries capable of detecting RNA transcripts and RNA splice variants which populate a transcriptome and which are transcribed from genes or transcription units that populate the corresponding genome. The present invention also provides oligonucleotide arrays generated from the oligonucleotide libraries and methods of using the oligonucleotide libraries in various oligonucleotide detection systems and expression profiling studies. Antisense molecules and double-stranded interfering RNAs, which are types of oligonucleotides, based on the oligonucleotides disclosed herein also are provided.

### CROSS REFERENCE TO RELATED APPLICATIONS

Incorporated by reference are U.S. Serial No. 60/287,724, filed May 2, 2001; U.S. Provisional Application Ser. No. 60/221,607, filed on Jul. 28, 2000; and U.S. application Ser. No. 09/133,987, filed on Aug. 13, 1998. This application claims priority to U.S. Serial No. 60/287,724, filed May 2, 2001 and U.S. Provisional Application Ser. No. 60/221,607, filed on Jul. 28, 2000.

### BACKGROUND OF THE INVENTION

1. Field of the Invention

The present invention provides oligonucleotide libraries that are useful for detecting messenger RNAs from a biological sample. More specifically, the present invention provides oligonucleotide libraries capable of detecting RNA transcripts, including RNA splice variants, which populate a transcriptome and which are transcribed from genes or transcription units that populate the corresponding genome. The present invention also provides oligonucleotide arrays generated from the oligonucleotide libraries and methods of using the oligonucleotide libraries in various oligonucleotide detection systems and expression profiling studies.

2. Description of the Field

Understanding biological functions and processes, normal or abnormal, in a cell, a tissue, an organ, and an organism hinges upon the knowledge of genes operative in the biological locale of interest, the messenger RNAs transcribed from these genes, and ultimately the set of proteins produced from the RNA transcripts. At the species level, the repertoire of all DNA molecules makes up the genome of that species; among these DNA molecules, the ones that are transcribed into RNA molecules are referred to as genes or transcription units. Correspondingly, the repertoire of all messenger RNA molecules transcribed from transcription units (hence “RNA transcripts” or “transcripts”) in a genome makes up a transcriptome; and, the repertoire of all proteins translated from messenger RNA molecules makes up a proteome.

Studies on a transcriptome of a species shed light on researchers' understanding of the corresponding genome and proteome of that species. Various techniques for identifying RNA transcripts and determining their levels of abundance in a biological sample have thus been instrumental in deciphering transcriptomes of a variety of species and thereby facilitating discoveries relating to the corresponding genomes and proteomes. These techniques include both gel-based procedures such as Northern Blot analysis, Dot Blot analysis, Primer Extension analysis, Substrate Enrichment Hybridization Analysis, Differential Display analysis, Polymerase Chain Reaction (PCR) based analysis and, most recently, chip-based procedures such as DNA microarray analysis.

The completion of whole genome sequencing for a number of species promises better efficiency, reliability, and comprehension in studying these genomes and their corresponding transcriptomes and proteomes. It is estimated that the total number of expressed genes or transcription units in a human genome is around 30,000-40,000 (Venter G. et al., Science 2000, Vol, 291: 1304-1351; The Genome International Sequencing Consortium, Nature 2000, Vol. 409: 360-921). However, the number of proteins encoded in a human proteome is expected to significantly exceed this estimate because, in many cases, more than one RNA splice variant is transcribed from a transcription unit or a gene. Estimates vary, but some researchers believe that a human transcriptome may contain up to 500,000 RNA transcripts and that, more than 30% of genes or transcription units in the human genome produce several RNA splice variants. (Mironov et al. 1999, Genome Research 9:1288-1293). These numbers are considered by others to be conservative. Alternative splicing also occurs in rat and mouse with similar frequencies, for example, and in lower organisms, such as Drosophila melanogaster and Caenorhabditis elegans. It should be noted that a special case of alternative splicing is alternative polyadenylation and that a significant number of genes or transcription units have alternative polyadenylation sites.

Proteins translated from different RNA splice variants may have significantly different biological functions. Different splice variants may be expressed in different tissues, different developmental stages, and different disease states. The detection of RNA transcripts and RNA splice variants from a transcription unit and the determination of (i) level of abundance of all RNA transcripts, including the splice variants from the transcription unit and (ii) levels of abundance of a subset of the splice variants or one splice variant are, therefore, desirable in accurately capturing the state of a transcriptome and hence, the corresponding proteome. And yet, the qualitative and quantitative detection of RNA splice variants remains an unmet challenge in the field of molecular biology, particularly on the scale of a transcriptome.

Oligonucleotide libraries have been used in both gel based and chip based systems to detect RNA molecules and measure their levels of abundance. However, oligonucleotides (“oligos”) of these libraries, which are used as oligo probes for hybridizing to target RNAs, are typically incapable of accurately detecting splice variants. That is, they typically do not contain oligos capable of hybridizing (thus recognizing) one or more specific splice variants, and therefore cannot effectively identify and/or distinguish alternatively spliced RNA transcripts or determine the levels of abundance of different splice variants.

### SUMMARY OF THE INVENTION

It is therefore an object of this invention to provide oligonucleotide libraries that are capable of detecting RNA transcripts and RNA splice variants transcribed from transcription units in a genome, thereby qualitatively and quantitatively characterizing the corresponding transcriptome. It is another object of this invention to provide oligonucleotide arrays that are generated from the oligonucleotide libraries and methods of using the oligonucleotide libraries to study a transcriptome of interest and, methods of using the oligonucleotide libraries in expression profiling studies.

In accordance with the present invention, there is provided, in an embodiment, an oligonucleotide library for detecting messenger RNAs that populate a transcriptome, wherein the transcriptome comprises messenger RNAs transcribed from a multiplicity of transcription units that populate a genome, wherein the library comprises a plurality of oligonucleotides, wherein each oligonucleotide in the plurality is capable of hybridizing selectively to a set of messenger RNAs transcribed from a given transcription unit of the genome, wherein at least one transcription unit of the genome encodes one or more messenger RNA splice variants.

According to another embodiment of the invention, the oligonucleotide library is capable of detecting RNA transcripts and splice variants of a human transcriptome. According to another embodiment, the oligonucleotide library of the present invention is capable of detecting RNA transcripts and splice variants of a rat transcriptome. According to yet another embodiment, the oligonucleotide library of this invention is capable of detecting RNA transcripts and splice variants of a mouse transcriptome. According to a further embodiment, the oligonucleotide library of this invention is capable of detecting RNA transcripts and splice variants of a arabadopsis transcriptome. According to a still further embodiment, the oligonucleotide library of this invention is capable of detecting RNA transcripts and splice variants of a drosophila melanogaster transcriptome.

In accordance with the present invention, there is provided, in another embodiment, an oligonucleotide library for detecting messenger RNAs that populate a sub transcriptome (a portion of a transcriptome that bears certain biological origin, structure, or function traits, e.g., tissue specificity, disease specificity, developmental specificity, etc.) of a tissue origin, wherein the sub transcriptome comprises messenger RNAs transcribed from a multiplicity of transcription units that populate a sub genome of the tissue origin, wherein the library comprises a plurality of oligonucleotides, wherein each oligonucleotide in the plurality is capable of hybridizing selectively to a set of messenger RNAs transcribed from a given transcription unit of the sub genome, wherein at least one transcription unit of the sub genome encodes one or more messenger RNA splice variants.

According to the invention, the tissue origin of the oligonucleotide library may be kidney, brain, heart, lung, bone, liver or other tissue of interest in various embodiments.

In accordance with the present invention, there is provided, in yet another embodiment, an oligonucleotide library for detecting messenger RNAs that populate a sub transcriptome of a pathological tissue origin, wherein the sub transcriptome comprises messenger RNAs transcribed from a multiplicity of transcription units that populate a sub genome (a portion of a genome that bears certain biological origin, structure, or function traits, e.g., tissue specificity, disease specificity, developmental specificity, etc.) of the pathological tissue origin, wherein the library comprises a plurality of oligonucleotides, wherein each oligonucleotide in the plurality is capable of hybridizing selectively to a set of messenger RNAs transcribed from a given transcription unit of the sub genome, wherein at least one transcription unit of the sub genome encodes one or more messenger RNA splice variants.

According to the invention, the pathological tissue origin may be cancer tissue, such as colon cancer tissue, breast cancer tissue, lung cancer tissue, bone cancer tissue, prostate cancer tissue, brain cancer tissue, or liver cancer tissue, in various embodiments. The pathological tissue origin, according to the invention, may also be abnormal heart tissue, abnormal neuronal tissue, abnormal liver tissue or abnormal kidney tissue in various embodiments, for example.

In accordance with the present invention, there is provided, in still another embodiment, an oligonucleotide library for detecting messenger RNAs that populate a sub transcriptome of a developmental stage, wherein the sub transcriptome comprises messenger RNAs transcribed from a multiplicity of transcription units that populate a sub genome of the developmental stage, wherein the library comprises a plurality of oligonucleotides, wherein each oligonucleotide in the plurality is capable of hybridizing selectively to a set of messenger RNAs transcribed from a given transcription unit of the sub genome, wherein at least one transcription unit of the sub genome encodes one or more messenger RNA splice variants.

According to the invention, the developmental stage may be human neural induction, mouse mesoderm induction, human erythrocyte differentiation, human or rat stem cell development, or other specific developmental biology states in various species.

In accordance with the present invention, there is provided, in a further embodiment, an oligonucleotide library for detecting messenger RNAs that populate a transcriptome of patients suffering from a disorder, wherein the transcriptome comprises messenger RNAs transcribed from a multiplicity of transcription units that populate a genome of patients suffering from the disorder, wherein the library comprises a plurality of oligonucleotides, wherein each oligonucleotide in the plurality is capable of hybridizing selectively to a set of messenger RNAs (one or more RNA transcripts) transcribed from a given transcription unit of the genome, wherein at least one transcription unit of the genome encodes one or more messenger RNA splice variants.

According to the invention, the disorder may be cancer, such as colon cancer, breast cancer, lung cancer, bone cancer, prostate cancer, brain cancer, or liver cancer, in various embodiments. The disorder, according to the invention, may also be Alzheimer's disease, Parkinson's disease, osteoporosis, diabetes, rheumatoid arthritis, or other disease of interest, in various embodiments.

In accordance with the present invention, there is provided, in a still further embodiment, an oligonucleotide library for detecting messenger RNAs that populate a transcriptome, wherein the transcriptome comprises messenger RNAs transcribed from a multiplicity of transcription units that populate a genome, wherein the library comprises a plurality of oligonucleotides, wherein each oligonucleotide in the plurality is capable of hybridizing selectively to one or a subset of messenger RNAs transcribed from a given transcription unit of the genome, wherein at least one transcription unit of the genome encodes one or more messenger RNA splice variants.

According to one embodiment of the invention, the oligonucleotide library is capable of detecting RNA transcripts and splice variants of a human transcriptome. According to another embodiment, the oligonucleotide library of the present invention is capable of detecting RNA transcripts and splice variants of a rat transcriptome. According to yet another embodiment, the oligonucleotide library of this invention is capable of detecting RNA transcripts and splice variants of a mouse transcriptome.

In accordance with the present invention, there is provided, in another embodiment, an oligonucleotide library for detecting messenger RNAs that populate a sub transcriptome of a tissue origin, wherein the sub transcriptome comprises messenger RNAs transcribed from a multiplicity of transcription units that populate a sub genome of the tissue origin, wherein the library comprises a plurality of oligonucleotides, wherein each oligonucleotide in the plurality is capable of hybridizing selectively to one or a subset of messenger RNAs transcribed from a given transcription unit of the sub genome, wherein at least one transcription unit of the sub genome encodes one or more messenger RNA splice variants.

According to the invention, the tissue origin of the oligonucleotide library may be kidney, brain, heart, lung, bone, liver or other tissue of interest in various embodiments.

In accordance with the present invention, there is provided, in yet another embodiment, an oligonucleotide library for detecting messenger RNAs that populate a sub transcriptome of a pathological tissue origin, wherein the sub transcriptome comprises messenger RNAs transcribed from a multiplicity of transcription units that populate a sub genome of the pathological tissue origin, wherein the library comprises a plurality of oligonucleotides, wherein each oligonucleotide in the plurality is capable of hybridizing selectively to one or a subset of messenger RNAs transcribed from a given transcription unit of the sub genome, wherein at least one transcription unit of the sub genome encodes one or more messenger RNA splice variants.

According to the invention, the pathological tissue origin may be cancer tissue, such as colon cancer tissue, breast cancer tissue, lung cancer tissue, bone cancer tissue, prostate cancer tissue, brain cancer tissue, or liver cancer tissue, in various embodiments. The pathological tissue origin, according to the invention, may also be abnormal heart tissue, abnormal neuronal tissue, abnormal liver tissue or abnormal kidney tissue in various embodiments.

In accordance with the present invention, there is provided, in still another embodiment, an oligonucleotide library for detecting messenger RNAs that populate a sub transcriptome of a developmental stage, wherein the sub transcriptome comprises messenger RNAs transcribed from a multiplicity of transcription units that populate a sub genome of the developmental stage, wherein the library comprises a plurality of oligonucleotides, wherein each oligonucleotide in the plurality is capable of hybridizing selectively to one or a subset of messenger RNAs transcribed from a given transcription unit of the sub genome, wherein at least one transcription unit of the sub genome encodes one or more messenger RNA splice variants.

According to the invention, the developmental stage may be human neural induction, mouse mesoderm induction, human erythrocyte differentiation, or other specific developmental states in various species.

In accordance with the present invention, there is provided, in a further embodiment, an oligonucleotide library for detecting messenger RNAs that populate a transcriptome of patients suffering from a disorder, wherein the transcriptome comprises messenger RNAs transcribed from a multiplicity of transcription units that populate a genome of patients suffering from the disorder, wherein the library comprises a plurality of oligonucleotides, wherein each oligonucleotide in the plurality is capable of hybridizing selectively to one or a subset of messenger RNAs transcribed from a given transcription unit of the genome, wherein at least one transcription unit of the genome encodes one or more messenger RNA splice variants.

According to the invention, the disorder may be cancer, such as colon cancer, breast cancer, lung cancer, bone cancer, prostate cancer, brain cancer, or liver cancer, in various embodiments. The disorder, according to the invention, may also be Alzheimer's disease, Parkinson's disease, osteoporosis, diabetes, rheumatoid arthritis, or other disease of interest, in various embodiments.

The oligonucleotide libraries of the present invention may have at least 95, 200, 400, 600, 800, or 1000 oligonucleotides in various embodiments. The oligenucleotides in the library of the present invention may be 75, 65, 60, 55, 50, 45, 40, 35, 30, or 20 bases in length, or any integer thereabout or therebetween, in various embodiments of the invention, although smaller and larger oligonucleotides may be employed according to the invention.

In accordance with the present invention, in a still further embodiment, each oligonucleotide of the aforementioned oligonucleotide libraries of the invention in various embodiments contains a modification that enables attachment to a solid surface. The attachment may be covalent or electrostatic, for example.

In accordance with the present invention, there is provided, in another embodiment, a DNA microarray having spotted thereon a plurality of oligonucleotide sequences, wherein said plurality is provided by the aforementioned oligonucleotide libraries of the invention in various embodiments.

In accordance with the present invention, there is provided, in yet another embodiment, a method for expression profiling a cell or tissue sample that contains two or more RNAs of various abundances, comprising measuring hybridization signals of said sample to a plurality of oligonucleotide sequences, thereby determining the levels of said two or more RNAs in said sample and, where a RNA is transcribed from a transcription unit that has a set of splice variants, determining (i) the total level of the set of splice variants, (ii) the total level of a subset thereof, or (iii) the level of one splice variant thereof, wherein said plurality is provided by the aforementioned oligonucleotide library the invention in various embodiments.

According to the invention, the method for expression profiling may, in various embodiments, employ a nucleotide chip, a membrane or filter, an electrophoresis gel and a filter or membrane imprinted therefrom, or other similar platforms, on which hybridization is carried out.

In accordance with yet another aspect of the invention, there are provided methods of inhibiting or preventing translation using the oligonucleotides of the present invention or DNA constructs based on the oligonucleotides, wherein the constructs can yield antisense RNA at the cellular level.

In accordance with still another aspect of the invention, the oligonucleotides of the libraries can be single stranded, double stranded or partially double stranded.

In accordance with a still further aspect of the invention, there are provided double stranded oligonucleotides that can cause post-transcriptional silencing of specific genes. In one embodiment, these oligonucleotides are short double-stranded RNAs

### BRIEF DESCRIPTION OF DRAWINGS

FIG. 1. This is a diagram demonstrating a transcription unit with three exons and, transcribed therefrom, two alternatively spliced transcripts.

FIG. 2. This is a diagram demonstrating a transcription unit with three exons and, transcribed therefrom, two different splice variants.

FIG. 3. This is a diagram demonstrating a transcription unit with five exons and, transcribed therefrom, four alternatively spliced transcripts.

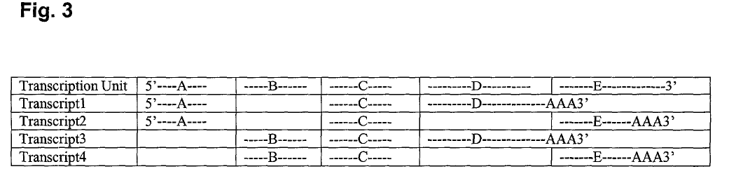

### DETAIL DESCRIPTIONS OF PREFERRED EMBODIMENTS

Libraries, Oligonucleotides, and Modifications Thereof

To characterize a transcriptome or a portion thereof, oligonucleotides used as probes to test hybridization to messenger RNA samples must be representative of the total population of RNA transcripts or a portion thereof that is of interest. To generate this type of oligonucleotide information, the approach outlined in U.S. patent application Ser. No. 09/133,987 (“the '987 application”) can be employed.

The large pool of publicly known nucleotide sequences, sequence fragments, or expressed sequence tags (“EST”), e.g., GenBank, EMBL, DBest sequence collections, are subject to rigorous clustering and assembly procedures to obtain sequence clusters and longer contigs (contiguous sequences, i.e., longer sequences assembled from shorter ESTs). These procedures give rise to sequences that are better representations of genes or transcription units compared to the large pool of ESTs and other sequence fragments.

A representative sequence for a given gene or transcription unit is chosen and from this sequence one or more oligonucleotide (or oligo, oligo probe) is derived. The representative sequence has high specificity to the target gene or transcription unit (i.e., it shares little homology to sequences from other genes or transcription unit therefore the frequency of non-specific binding is minimized); the sequence quality is good (i.e., the gene sequence information is accurate). The oligonucleotide probe has high sensitivity, i.e., the affinity of hybridization of the probe to the target gene or transcription unit is high. The probe preferably recognizes a sequence close to the 3′ terminus of the target gene or transcription unit such that in certain procedures where target gene sample is prepared the yield of which is maximized. Additionally, the oligonucleotide lacks secondary structure (e.g., hairpin) or tendency to form the same, such that hybridization is not hampered.

Most importantly, a representative sequence is chosen and one or more oligonucleotides are designed taking into account alternative splicing of the target gene and transcription unit. As such, the oligonucleotide may be used to hybridize, and hence detect, either (i) all the splice variants of a particular gene or transcription unit, (ii) a subset of all the splice variants, or (iii) one of the splice variant of the gene or transcription unit.

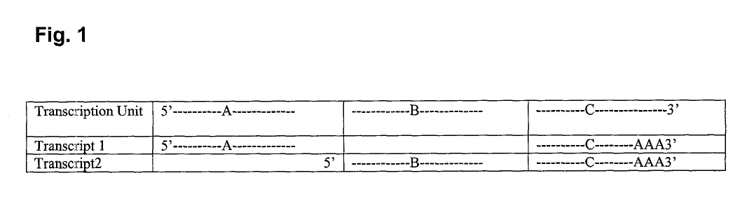

Referring to FIG. 1, a transcription unit or gene has, for example, three exons: A, B, and C, and is alternatively spliced in transcription giving rise to two variants: transcript 1 (AC) and transcript 2 (BC). An oligo complimentary to sequence A, or a fragment thereof, would therefore only detect transcript 1, not transcript 2. An oligo complimentary to sequence B or a fragment thereof, by contrast, would only detect transcript 2, not transcript 1.

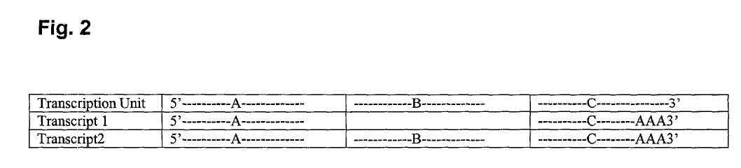

The scenario shown in FIG. 2 is slightly different. The transcription unit has three exons: A, B, and C, which are transcribed into two splice variants, transcript 1 (AC) and transcript 2 (ABC). An oligo complementary to sequence A or a fragment thereof would be able to detect both transcript 1 and transcript 2 and, collectively, measure the level of abundances of both. In contrast, the oligo complementary to sequence B or a fragment thereof would be able to detect transcript 2 not transcript 1, thereby providing direct information on the abundance of transcript 2.

FIG. 4 demonstrates a transcription unit or a gene that has 5 exons: A, B, 20 C, D, and E. There are four alternatively spliced transcripts: transcript 1 (ACD), 2 (ACE), 3 (BCD), and 4 (BCE). This is a more complex scheme but the principle remains the same: An oligo complementary to sequence A or a fragment thereof would be able to only detect transcripts 1 and 2 (hence “1+2=A”); an oligo complementary to sequence B would be able to only detect transcripts 3 and 4 (hence “3+4=B”); an oligo complementary to sequence D would be able to only detect transcripts 1 and 3 (hence “1+3=D”); and an oligo complimentary to sequence E would be able to only detect transcripts 2 and 4 (hence “2+4=E”) Therefore, we have a number of oligos that can specifically detect a subset of the splice variants. And, additionally, resolving the four polyvariance equations above would reveal the level of abundance of each splice variant (A, B, D, and E). Moreover, an oligo complementary to sequence C or a fragment thereof would be able to detect all four transcripts and thus measure the total level of abundances of these splice variants.

Analogously, according to the present invention, alternative polyadenylation is taken into account such that a representative sequence and one or more oligonucleotide probe are generated for a particular gene or transcription unit that has alternative polyadenylation sites. These probes may be used to hybridize, and hence detect, either (i) all the alternative polyadenylated transcripts, (ii) a subset of these alternative polyadenylated transcripts, or (iii) one of the polyadenylated sites of the target gene or transcript.

The oligonucleotides of the present invention may be modified at the termini to facilitate their application in a gel-based system or attachment on a chip or array-based system for RNA detection. For example, in one embodiment, the oligonucleotides are modified at the 5′ terminus: A 5′ C6-amino modification is performed to enable covalent attachment of oligonucleotides on a glass array surface. The carbons function as a spacer and the reactive amine group interacts with aldehydes that are covalently attached to a glass surface. Pre-processed glasses are commercially available from a number of vendors, e.g., ArrayIt.com (http://arrayit.com), Corning (http://www.corning.com), Clontech (http://www.clontech.com). These glasses, and other similar ones, are suitable for oligonucleotide binding and thereby for making oligo arrays using the oligonucleotide libraries according to this invention. The attachment may be covalent or electrostatic, for example. One example is binding of oligos on poly-L-lysine glass, which is electrostatic: poly-L-lysine is positively charged whereas phosphate backbone of DNA is negatively charged. Another example is binding of oligos on aldehyde glass, which is covalent.

Oligonucleotides Used with Gel-Based and Array-Based Platforms

As mentioned above, various detection platforms may be used according to the present invention to measure RNA species in a transcriptome. In one embodiment, a gel-based platform is used; in another embodiment, a chip or array-based platform is used. When a gel-based platform is used, techniques such as Northern Blot analysis, Dot Blot analysis, Primer Extension analysis, Substrate Enrichment Hybridization Analysis, Differential Display analysis, Polymerase Chain Reaction (PCR) based analysis, or other similar procedures may be employed. Arrays also can be made by spotting directly on a substrate like nitrocellulose. Essentially, oligonucleotides of this invention are labeled (via radioactive-labeling, fluorescent-labeling, or using other suitable labeling methods and capture moieties known in the art) and are hybridized to a filter or membrane imprinted from the gel in which RNA or cDNA samples have been run. Hybridization signals indicate the identity and abundance of the RNA transcripts (and splice variant(s) thereof) that the oligonucleotide probes represent. These gel-based nucleotide hybridization and detection procedures are well known to a skilled molecular biologist. See generally, Sambrook J. and Russell, 2001, Molecular Cloning, a Laboratory Manual, 3rd Ed. In some cases, where separation of nucleotides is not involved, nucleotide probes and samples may be directly applied to a membrane or filter for hybridization without the use of an electrophoresis gel.

When certain chip or array-based platforms are used, the oligonucleotides of the present invention are spotted or printed on a solid surface, e.g., a glass slide, via various attachment chemistries as discussed above or via other suitable attachment procedures. The slide is then treated in a manner similar to a filter or membrane in a typical gel-based separation and hybridization experiment and, subsequently, probed with labeled (via radioactive-labeling, fluorescent-labeling, or by other suitable labeling methods) cDNA or RNA molecules that are derived from a biological sample such as a tissue or cell line of interest. In some cases, labeled clones or total genomic DNA may be used instead. Following hybridization, the slide is washed and scanned to read and record the hybridization signals. These signals represent the identity and abundance of the RNA transcripts (and splice variant(s) thereof) that the oligonucleotide probes correspond to and detect. Such measurement of RNA transcripts and their splice variants in a particular biological sample therefore provides a snapshot of the transcriptome under a particular biological state—normal, pathological, or treated—and hence helps to elucidate the characteristics and interactions in different biological states.

According to the invention all oligonucleotides of a library, or a portion thereof, may be spotted on a single microarray and, therefore, a single hybridization procedure may test a multiplicity of transcripts or their corresponding genes. As a variation, in another embodiment, oligonucleotides may be directly synthesized on the surface of an array. Methods of oligo synthesis on a solid surface are known in the art. See, e.g., U.S. Pat. No. 5,593,839. In this embodiment, the oligonucleotides in the library preferably are around 20 or 25 bases in length, since in situ synthesis of longer sequences is often error prone. The libraries of the present invention that are directly synthesized on an array thus may be useful as specialized or mini libraries designed to detect transcripts of a sub-transcriptome under a particular biological state.

Uses for Oligonucleotide Libraries

Whether gel-based, array-based, or other suitable detection systems are employed, according to the present invention, the oligonucleotide library may be constructed to detect RNA transcripts and splice variants of the transcriptome of a given species, such as a human transcriptome, a mouse transcriptome, or a rat transcriptome. The library also may be constructed to detect RNA transcripts and splice variants of a sub-transcriptome that is of a specific biological or pathological origin or in a specific biological or pathological state.

For example, in one embodiment of this invention, the sub-transcriptome is of a specific tissue origin. That is, it may be a kidney sub-transcriptome, a brain tissue sub-transcriptome, a heart tissue sub-transcriptome, a lung tissue sub-transcriptome, a bone tissue sub-transcriptome, a liver tissue sub-transcriptome, etc. The libraries of the invention can thus be used to specifically detect RNA transcripts and splice variants that populate the sub-transcriptome of a tissue of interest. Tissue-specific expression of certain genes (i.e., certain RNA transcripts or splice variants that exist only in some sub-transcriptome(s) but not in others) or differential expression of certain genes in a tissue-specific manner (i.e., certain RNA transcripts or splice variants are more—or less—abundant in some sub-transcriptome(s) than in another) therefore can be detected and monitored using the oligonucleotide library of the present invention. The presence or absence of a transcript or specific splice variant and the differences in their abundance may be evaluated under a statistical significance standard. Suitable statistical evaluations are known in the art.

In another embodiment, the sub-transcriptome is of a particular pathological origin. That is, it may be a cancer tissue sub-transcriptome, e.g., a colon cancer tissue sub-transcriptome, a breast cancer tissue sub-transcriptome, a lung cancer tissue sub-transcriptome, a bone cancer tissue sub-transcriptome, a prostate cancer tissue sub-transcriptome, a brain cancer tissue sub-transcriptome, or a liver cancer tissue sub-transcriptome. It may also be an abnormal heart tissue sub-transcriptome, an abnormal neuronal tissue sub-transcriptome, an abnormal liver tissue sub-transcriptome, or an abnormal kidney tissue sub-transcriptome, etc.

The libraries of the invention can therefore be used to specifically detect RNA transcripts and splice variants that populate the sub-transcriptome of a pathological origin of interest. This application, therefore, allows detection of tissue- and pathology-specific genes, i.e., genes that are only expressed in a specific tissue and under a specific pathological condition, since the corresponding transcripts and splice variants can only be found in the specific sub-transcriptome. Additionally, this application further permits detection of genes, that are differentially expressed in a tissue- and pathology-specific manner, i.e., genes that are expressed at a different level in a specific tissue and under a specific pathological condition than in other tissues or under normal or other conditions, because the corresponding transcripts and splice variants are more—or less—abundant in the specific sub-transcriptome. The presence or absence of a transcript or splice variant and the differences in their abundance may be evaluated under a statistical significance standard.

In yet another embodiment, the sub-transcriptome is of a certain developmental stage. That is, it may be a sub-transcriptome from human neural induction, mouse mesoderm induction, human erythrocyte differentiation, or other specific developmental biology states in various species. The libraries of the invention can therefore be used to specifically detect RNA transcripts and splice variants that populate the sub-transcriptome of a developmental biology stage of interest. This application allows detection of developmental specific genes, i.e., genes that are only expressed in a specific developmental state, since the corresponding transcripts and splice variants can only be found in the specific sub-transcriptome. Additionally, this application also allows detection of genes that are differentially expressed in a developmental-dependent manner, i.e., genes that are expressed at a different level in a specific developmental state than in other state, because the corresponding transcripts and splice variants are more—or less—abundant in the specific sub-transcriptome. The presence and absence of a transcript or splice variant and the differences in their abundance may be evaluated under a statistical significance standard.

In a further embodiment, the oligonucleotide library is constructed to detect RNA transcripts and splice variants of a transcriptome of a patient suffering from a particular disorder. That is, it may be the transcriptome of a cancer patient, e.g., a colon cancer patient, a breast cancer patient, a lung cancer patient, a bone cancer patient, a prostate cancer patient, a brain cancer patient, or a liver cancer patient. It may also be the transcriptome of an Alzheimer patient, a Parkinson's patient, an osteoporosis patient, a diabetes patient, a rheumatoid arthritis, or a patient of other disease of interest.

The present invention allows detection of disease specific genes, i.e., genes that are only expressed in patients afflicted with a specific disease, since the corresponding transcripts and splice variants can only be found in the transcriptome of such patients. Additionally, the present invention allows detection of genes that are differentially expressed in patients of a specific disease, i.e., genes that are expressed at a different level in patients of a specific disease than in normal or healthy individuals, because the corresponding transcripts and splice variants are more—or less—in the transcriptome of such patients. The presence and absence of a transcript or splice variant and the differences in their abundance may be evaluated under a statistical significance standard, e.g., requiring a specific p score. Furthermore, this application allows individualized characterization of expression patterns or profiles (the compilation of the identity and abundance of transcripts and splice variants of a given patient) of patients suffering from a particular disease and therefore provides a basis for development of individualized therapeutics.

To summarize, the oligonucleotide libraries of the present invention may be used in a variety of functional contexts to detect RNA transcripts and splice variants and thereby characterize a transcriptome or sub-transcriptome of interest. In this connection, the scope of the sub-transcriptome may also be delimited by protein functions. For example, an oligonucleotide library may be used to detect RNA transcripts and splice variants of a sub-transcriptome that corresponds to cell surface antigens. A list of more than 40 protein functional groups, as set forth below (see also, U.S. Provisional Application Ser. No. 60/221,607), is useful in the present invention, for example, for constructing oligonucleotide libraries capable of characterizing sub-transcriptomes that give rise to sub-proteomes or proteins of specific functions and abnormality thereof.

Group 1. Adaptor-binding proteins. These are proteins that are associated to other cell components—binding to or interacting with—in maintaining their structural integrity and performing its functional activities.

Group 2. Adhesion molecules. These are proteins involved in modulation of adhesion between adjoining cells.

Group 3. Apolipoproteins. These are proteins that are part of lipoprotein particles and that function in cellular signaling in binding and internalization of these particles. Apolipoprotein defects are found in diseases which involve abnormally high or low levels of lipoprotein and cholesterol, as well as conditions involved in the formation or arteriosclerosis.

Group 4. Apoptosis related proteins. These are proteins and enzymes that are involved in the apoptosis pathway, in an inhibitory or stimulatory manner. Abnormalities of these proteins cause diseases which are involved in premature death of cells, such as degenerative diseases, for example neurodegenerative diseases or conditions associated with aging, or alternatively, diseases wherein required apoptosis does not take place. Example of such diseases are cancerous diseases and loss of cardiac function after myocardial infarction.

Group 5. Cancer related proteins. These proteins, including DNA repair proteins, tumor markers and antigens, tumor suppressors, and messenger molecules participating tumorigenesis, etc., are involved in various kind of cancers and the treatment and detection of the corresponding metastasis states.

Group 6. Carboxylases. Abnormality of these proteins causes mal-regulation of enzymatic reactions that remove CO₂ groups from other moieties.

Group 7. Cell surface antigens. Proteins expressed on the surface of cells. Abnormalities of these proteins can be seen in autoimmune disease, e.g., AIDS and cancers that involve cell surface antigens, for example.

Group 8. Proteins controlling cell growth. These proteins have defective structure or function in degenerative diseases (low growth) or cancerous diseases (uncontrolled growth).

Group 9. Coagulation—related proteins. Abnormality of these proteins may cause hemophilia or stroke and blockage of blood vessels, for example.

Group 10. Converting enzymes. These enzymes convert one protein to another by specific cleavage of the precursor protein.

Group 11. Cyclase enzymes. These enzymes convert triphosphate to cyclic monophosphate. Abnormality of these enzymes causes insufficient or excessive conversion of triphosphate to cyclic monophosphate thereby affecting cellular signaling.

Group 12. Proteins involved in protein degradation. Abnormalities of these proteins may cause abnormal degradation of other proteins, which may lead to abnormal accumulation of various proteinaceous product in cells.

Group 13. Proteins involved in development. Abnormalities of these proteins are manifested in genetic diseases involving abnormal development of a fetus, for example.

Group 14. Domain proteins. These are proteins that are involved in protein-protein interactions.

Group 15. Esterase. These proteins cleave an ester bond.

Group 16. Growth factors. Examples of these proteins include cytokines, interleukins, interferon, and lymphokines, etc. These proteins are implicated in autoimmune diseases, inflammation related disease, Graft vs. Host disease, diseases caused by infectious agents, and cancer, to name a few.

Group 17. Hormones and poietin proteins. Abnormality of these proteins are seen in various endocrine disorders.

Group 18. Housekeeping proteins. Examples are homeobox proteins, heat shock proteins, and chaperoning, etc.

Group 19. Hydrolases. These enzymes modify hydroxyl groups; examples are hydrogenase, dehydrogenase, hydrolase, and hydroxylase.

Group 20. Immunology-related genes. These are proteins that are involved in the immune system, including antigens, antibodies, and their associated proteins. These proteins are important in pathological conditions such as inflammation, autoimmune diseases, infectious diseases, and cancerous processes.

Group 21. Inhibitors. These proteins inhibit the function of other proteins in cellular processes.

Group 22. Kinases. Abnormality of these proteins may lead to defective cellular signaling.

Group 23. Lipases, phospholipases, and lysophospholipases. These proteins are implicated in abnormal lipid metabolism.

Group 24. Cell matrix and cytoskeleton proteins.

Group 25. Modifying enzymes. These proteins include a number of miscellaneous enzymes such as paraoxonase, GTPase, ATPase, and anhydrase. Malfunctions of these enzymes are implicated in a variety of cellular processes.

Group 26. Mutases and superoxidedismutases. These proteins are implicated in cancer and various other pathological processes involved in aging.

Group 27. Neurology related proteins. These proteins are involved in central nervous system disorders, including various types of dementia, neurodegenerative diseases, epilepsy, psychiatric disorders, etc.

Group 28. Oxidases and peroxidases. Abnormalities of these enzymes may cause metabolic problems involving peroxide, for example.

Group 29. Oxygenases, mono- and di-oxygenases.

Group 30. Phosphatases and phosphorylases.

Group 31. Phosphoproteins and phospholipids.

Group 32. Proteases, peptidases, and proteinases.

Group 33. Receptors.

Group 34. Reductases.

Group 35. Secreted proteins. These proteins include hormones, neurotransmitters, and various other proteins secreted by cells to the extracellular environment.

Group 36. Signal-transduction proteins. A G protein is one example of this group of proteins.

Group 37. Sub-cellular proteins. Examples of these proteins include ribosomal proteins.

Group 38. Synthases and synthetases.

Group 39. Proteins involved in nucleotide interactions. These include transcription factors, RNA and DNA binding proteins, zinc fingers, helicase, isomerase, histones, nucleases.

Group 40. Transferases. These are enzymes involved in transfer of protein functional groups.

Group 41. Translational-factors. These are proteins and enzymes involved in the translational process, such as elongation and initiation factors. Abnormalities in these proteins may impair cellular protein production.

Group 42. Transporters. These proteins mediate the transport of molecules and macromolecules, including channels, exchangers, and pumps.

Expression Profiling Using Oligonucleotide Libraries

One application of the oligonucleotide libraries according to this invention is expression profiling, which is mentioned supra in various places but which warrants some additional discussion. Expression profiling, also termed gene expression profiling, as used in this invention, means quantitative and qualitative determination of RNA transcripts and splice variants in a biological sample. The biological sample may be a tissue or a cell line sample, a normal or diseased tissue sample, a diseased tissue sample or a tissue sample after some period of treatment, a sample taken from different developmental biology state, a biological tissue or fluid sample taken from a patient at various time points in the course of application of a drug or a treatment regime. Expression profiling establishes expression profiles or expression patterns, which are compilations of identity and abundance of RNA transcripts and slice variants, for a particular biological source or a particular physiological or pathological state.

Expression profiling may be performed via a gel (and/or membrane)-based or array-based system according to the present invention, as discussed supra. Essentially, expression profiling of a biological sample that contains two or more RNAs of various abundances is preformed by measuring hybridization signals of the sample to a plurality of oligonucleotide sequences, thereby determining the levels of the RNA transcripts in the sample and, where an RNA is transcribed from a transcription unit that has a set of splice variants, determining (i) the total level of the set of splice variants, (ii) the total level of a subset thereof, or (iii) the level of one splice variant thereof. The plurality of oligonucleotides is provided by the oligonucleotide library of the present invention in various embodiments discussed above.

Accordingly, the present invention encompasses oligonucleotide libraries and sub-libraries thereof, custom or modified oligonucleotides, oligonucleotide arrays having spotted thereon the oligonucleotides from the libraries, methods of using oligonucleotides in various nucleotide detection systems, and methods of expression profiling using the oligonucleotide libraries.

Antisense Uses and RNA Interference

Because the oligonucleotides of the present invention are able to bind to one or more splice variants of the transcriptome, these oligonucleotides have uses in in situ and in vivo antisense contexts. For example, single-stranded antisense DNA oligonucleotides themselves, or double stranded DNA oligonucleotides in denaturing contexts, can be injected into a cell, such as a mammalian cell (including human cells) and the body, such as a mammalian body (including humans), and be expected to bind to mRNA transcripts, which would inhibit or prevent translation of the mRNA into protein. Moreover, the sense strand of an double-stranded oligonucleotide or a single-stranded sense oligonucleotide can be used as an initial design template for creation of antisense RNA. Using approaches known in the art for in vivo expression of antisense RNA molecules, an antisense RNA based upon the oligonucleotides of the present invention can be employed to inhibit or prevent translation of an mRNA at the cellular level.

The oligonucleotides of the library also can be employed in an RNA interference context. The phenomenon of RNA interference is discussed in Bass, Nature 411: 428-29 (2001); Elbahir et al., Nature 411: 494-98 (2001); and Fire et al., Nature 391: 806-11 (1998), where methods of making interfering RNA also are discussed. The double-stranded RNA based upon a sequence disclosed herein is less than 100 base pairs (“bps”) in length and constituency and preferably is about 30 bps or shorter, and can be made be approaches known in the art, including the use of complementary DNA strands or synthetic approaches. The RNAs that are capable of causing interference can be referred to a small interfering RNAs (“siRNA”), and can cause cause post-transcriptional silencing of specific genes in cells, such as mammalian cells (including human cells) and in the body, such as mammalian bodies (including humans). Exemplary siRNAs according to the invention could have up to 29 bps, 25 bps, 22 bps, 21 bps, 20 bps, 15 bps, 10 bps or any number thereabout or therebetween.

### EXAMPLE 1

### Oligonucleotide Libraries for Detecting all Alternatively Spliced Transcripts: Human, Rat, and Mouse Libraries

Oligonucleotides either 60 bases or 65 bases in length are synthesized, each of which is modified at the 5′ terminus with a C6-amino addition to enable subsequent covalent attachment to aldehyde glass surface. Each oligonucleotide is complementary to a portion of a RNA transcript that is common among the existing RNA splice variants transcribed from a particular transcription unit or gene. Each oligonucleotide is also specific or unique to the RNA transcripts and/or the RNA splice variants to which it is complementary, i.e., its binding to RNA transcripts or splice variants from other transcription units or genes is negligible. In other words, a single oligo is selected for each gene or transcription unit. Each oligo is derived from a sequence segment that is common to a practically maximal number of splice variants known or predicted for each gene or transcription unit. Additionally, the oligos are designed and synthesized in such a manner that high accuracy of sequence quality is maintained by avoiding sequencing errors, that the secondary structure is avoided to promote effective hybridization, and that the melting temperature is normalized to be consistent across the entire oligo collection in the library.

In some representative embodiments, the human oligo library comprises 18,861 human oligonucleotides, each 60 bases in length; the rat oligo library comprises 4,854 rat oligonucleotides, each 65 bases in length; and the mouse oligo library comprises 7,524 oligonucleotides, each 65 bases in length. The sequences of the 18,861 human oligos, the 4,854 rat oligos, and the 7,524 mouse oligos are contained in CRF, PC/MS-DOS PATENTIN 3.0, and copies comprising 32,337 sequences in Patentin 3.0 filed along with this application on CD-R media, file size 5.78 MB, file name Shoshan, with Attorney Docket No. 36688-0005, the entirety of which is hereby incorporated by reference, which contains the sequences set forth in the Sequence Listing (CRF and copies) comprising 32,337 sequences in Patentin 3.0 filed along with this application on CD-R media, file size 5.78 MB, file name Shoshan, with Attorney Docket No. 36688-0004, the entirety of which is hereby incorporated by reference, which was filed in U.S. Serial No. 60/287,724, filed May 2, 2001. Also provided herewith are the 32,337 sequences used in the sequence listing, which are in a MS Word format on a CD-R disk labeled Raw Sequences, PC/MS-DOS PATENTIN 3.0 for the 5.78 MB sequence listing, with Attorney Docket No. 36688-0005, the entirety of which is hereby incorporated by reference. Each oligo in these libraries represents a RNA transcript and/or all existing or predicted RNA splice variants that are transcribed from a transcription unit or gene encoding a certain functional protein. The name, GenBank accession number, and other annotative information of the corresponding gene or transcription unit for the oligo sequences in many cases are specified. The number of sequences above are not absolute, and therefore other libraries can be made according to applicants' teachings that contain different numbers of sequences.

The oligos of the library may be arranged on plates, e.g., in 384 or 96 well format. They are made spot or print-ready, and thus can conveniently be applied in microarray analysis.

Oligonucleotide libraries for other species, such as bovine, porcine, or arabadopsis may be similarly constructed according to the present invention; those libraries may be readily used in microarray analysis and, in other RNA or cDNA detection systems known in the art.

### EXAMPLE 2

### Mini Oligonucleotide Library for Detecting all RNA Transcripts or Distinct Splice Variants

Human oligo mini-libraries are constructed for human cancer/apoptosis genes, obesity/diabetes genes, and toxicology genes. Oligonucleotides 60 bases in length are designed or selected for genes implicated in cancer/apoptosis, obesity/diabetes, and toxicology, respectively, using approaches outlined in U.S. application Ser. No. 09/133,987, also taking into account the phenomenon of alternative splicing. These oligos are then synthesized taking into account the similar considerations as discussed in Example 1 supra.

The oligonucleotides in the human oligo mini-library capable of detecting distinct splice variants of human cancer/apoptosis genes and/or their transcripts comprise 201 sequences. The oligonucleotides in the human oligo mini-library capable of detecting all existing or predicted splice variants from human cancer/apoptosis genes comprise 280 sequences. The oligonucleotides in the human oligo mini-library capable of detecting distinct splice variants of genes implicated in toxicology comprise 234 sequences; and the oligonucleotides in the human oligo mini-library capable of detecting all existing or predicted splice variants from human toxicology genes comprise 96 sequences. The oligonucleotides in the human oligo mini-library capable of detecting distinct splice variants of human obesity/diabetes genes comprise 195 sequences; and the oligonucleotides in the human oligo mini-library capable of detecting all existing or predicted splice variants from human obesity/diabetes genes comprise 92 sequences. Again, the number of sequences above are not absolute, and therefore other libraries can be made according to applicants' teachings that contain different numbers of sequences.

The sequences of all the oligos in the six mini-libraries are enclosed in the Sequence Listing filed along with this application. These oligos may be arranged on plates, e.g., in 384 or 96 well format. They are made print-ready, and thus can conveniently applied in microarray analysis. Disease specific or differentially expressed genes can therefore be detected or monitored using these oligo libraries and microarrays for human toxicology, diabetes/obesity, and cancer/apoptosis conditions, respectively.

It is to be understood that while the invention has been described in detail by way of example and illustration for the purpose of clarity of teaching, the foregoing description is not intended to limit the scope of the invention. Other aspects, advantages, and modifications that are apparent to one of skill in the art in light of the teachings of this invention are within the scope of the following claims.

What is claimed is:

1. An oligonucleotide library for detecting messenger RNAs that populate a transcriptome, wherein the transcriptome comprises messenger RNAs transcribed from a multiplicity of transcription units that populate a genome, wherein the library comprises a plurality of oligonucleotides, wherein each oligonucleotide in the plurality is capable of hybridizing selectively to a set of messenger RNAs transcribed from a given transcription unit of the genome, wherein at least one transcription unit of the genome encodes one or more messenger RNA splice variants.

2. The oligonucleotide library of claim 1, wherein said transcriptome is a human transcriptome.

3. The oligonucleotide library of claim 1, wherein said transcriptome is a rat transcriptome.

4. The oligonucleotide library of claim 1, wherein said transcriptome is a mouse transcriptome.

5. An oligonucleotide library for detecting messenger RNAs that populate a sub transcriptome of a tissue origin, wherein the sub transcriptome comprises messenger RNAs transcribed from a multiplicity of transcription units that populate a sub genome of the tissue origin, wherein the library comprises a plurality of oligonucleotides, wherein each oligonucleotide in the plurality is capable of hybridizing selectively to a set of messenger RNAs transcribed from a given transcription unit of the sub genome, wherein at least one transcription unit of the sub genome encodes one or more messenger RNA splice variants.

6. An oligonucleotide library for detecting messenger RNAs that populate a sub transcriptome of a pathological tissue origin, wherein the sub transcriptome comprises messenger RNAs transcribed from a multiplicity of transcription units that populate a sub genome of the pathological tissue origin, wherein the library comprises a plurality of oligonucleotides, wherein each oligonucleotide in the plurality is capable of hybridizing selectively to a set of messenger RNAs transcribed from a given transcription unit of the sub genome, wherein at least one transcription unit of the sub genome encodes one or more messenger RNA splice variants.

7. The oligonucleotide library of claim 6, wherein said pathological tissue origin is cancer tissue.

8. An oligonucleotide library for detecting messenger RNAs that populate a sub transcriptome of a developmental stage, wherein the sub transcriptome comprises messenger RNAs transcribed from a multiplicity of transcription units that populate a sub genome of the developmental stage, wherein the library comprises a plurality of oligonucleotides, wherein each oligonucleotide in the plurality is capable of hybridizing selectively to a set of messenger RNAs transcribed from a given transcription unit of the sub genome, wherein at least one transcription unit of the sub genome encodes one or more messenger RNA splice variants.

9. An oligonucleotide library for detecting messenger RNAs that populate a transcriptome of patients suffering from a disorder, wherein the transcriptome comprises messenger RNAs transcribed from a multiplicity of transcription units that populate a genome of patients suffering from the disorder, wherein the library comprises a plurality of oligonucleotides, wherein each oligonucleotide in the plurality is capable of hybridizing selectively to a set of messenger RNAs transcribed from a given transcription unit of the genome, wherein at least one transcription unit of the genome encodes one or more messenger RNA splice variants.

10. The oligonucleotide library of claim 9, wherein said disorder is cancer.

11. A DNA microarray having spotted thereon a plurality of oligonucleotide sequences, wherein said plurality is provided by the oligonucleotide library of claim 1 or a subset thereof.

12. A DNA microarray having spotted thereon a plurality of oligonucleotide sequences, wherein said plurality is provided by the oligonucleotide library of claim 5 or a subset thereof.

13. A DNA microarray having spotted thereon a plurality of oligonucleotide sequences, wherein said plurality is provided by the oligonucleotide library of claim 6 or a subset thereof.

14. A DNA microarray having spotted thereon a plurality of oligonucleotide sequences, wherein said plurality is provided by the oligonucleotide library of claim 8 or a subset thereof.

15. A DNA microarray having spotted thereon a plurality of oligonucleotide sequences, wherein said plurality is provided by the oligonucleotide library of claim 9 or a subset thereof.

16. A method for expression profiling a cell or tissue sample that contains two or more RNAs of various abundances, comprising measuring hybridization signals of said sample to a plurality of oligonucleotide sequences, thereby determining the levels of said two or more RNAs in said sample and, where a RNA is transcribed from a transcription unit that has a set of splice variants, determining the total level of the set of splice variants, wherein said plurality is provided by the oligonucleotide library of claim 1 or a subset thereof.

17. The method of claim 16, wherein said hybridization signals are obtained from a nucleotide chip.

18. The method of claim 16, wherein said hybridization signals are obtained from an electrophoresis gel.

19. A method for expression profiling a cell or tissue sample that contains two or more RNAs of various abundances, comprising measuring hybridization signals of said sample to a plurality of oligonucleotide sequences, thereby determining the levels of said two or more RNAs in said sample and, where a RNA is transcribed from a transcription unit that has a set of splice variants, determining the total level of the set of splice variants, wherein said plurality is provided by the oligonucleotide library of claim 5 or a subset thereof.

20. The method of claim 19, wherein said hybridization signals are obtained from a nucleotide chip.

21. The method of claim 19, wherein said hybridization signals are obtained from an electrophoresis gel.

22. A method for expression profiling a cell or tissue sample that contains two or more RNAs of various abundances, comprising measuring hybridization signals of said sample to a plurality of oligonucleotide sequences, thereby determining the levels of said two or more RNAs in said sample and, where a RNA is transcribed from a transcription unit that has a set of splice variants, determining the total level of the set of splice variants, wherein said plurality is provided by the oligonucleotide library of claim 6 or a subset thereof.

23. The method of claim 22, wherein said hybridization signals are obtained from a nucleotide chip.

24. The method of claim 22, wherein said hybridization signals are obtained from an electrophoresis gel.

25. A method for expression profiling a cell or tissue sample that contains two or more RNAs of various abundances, comprising measuring hybridization signals of said sample to a plurality of oligonucleotide sequences, thereby determining the levels of said two or more RNAs in said sample and, where a RNA is transcribed from a transcription unit that has a set of splice variants, determining the total level of the set of splice variants, wherein said plurality is provided by the oligonucleotide library of claim 8 or a subset thereof

26. The method of claim 25, wherein said hybridization signals are obtained from a nucleotide chip.

27. The method of claim 25, wherein said hybridization signals are obtained from an electrophoresis gel.

28. A method for expression profiling a cell or tissue sample that contains two or more RNAs of various abundances, comprising measuring hybridization signals of said sample to a plurality of oligonucleotide sequences, thereby determining the levels of said two or more RNAs in said sample and, where a RNA is transcribed from a transcription unit that has a set of splice variants, determining the total level of the set of splice variants, wherein said plurality is provided by the oligonucleotide library of claim 9 or a subset thereof.

29. An oligonucleotide library for detecting messenger RNAs that populate a transcriptome, wherein the transcriptome comprises messenger RNAs transcribed from a multiplicity of transcription units that populate a genome, wherein the library comprises a plurality of oligonucleotides, wherein each oligonucleotide in the plurality is capable of hybridizing selectively to one or a subset of messenger RNAs transcribed from a given transcription unit of the genome, wherein at least one transcription unit of the genome encodes one or more messenger RNA splice variants.

30. The oligonucleotide library of claim 29, wherein said transcriptome is a human transcriptome.

31. The oligonucleotide library of claim 29, wherein said transcriptome is rat transcriptome.

32. The oligonucleotide library of claim 29, wherein said transcriptome is a mouse transcriptome.

33. An oligonucleotide library for detecting messenger RNAs that populate a sub transcriptome of a tissue origin, wherein the sub transcriptome comprises messenger RNAs transcribed from a multiplicity of transcription units that populate a sub genome of the tissue origin, wherein the library comprises a plurality of oligonucleotides, wherein each oligonucleotide in the plurality is capable of hybridizing selectively to one or a subset of messenger RNAs transcribed from a given transcription unit of the sub genome, wherein at least one transcription unit of the sub genome encodes one or more messenger RNA splice variants.

34. An oligonucleotide library for detecting messenger RNAs that populate a sub transcriptome of a pathological tissue origin, wherein the sub transcriptome comprises messenger RNAs transcribed from a multiplicity of transcription units that populate a sub genome of the pathological tissue origin, wherein the library comprises a plurality of oligonucleotides, wherein each oligonucleotide in the plurality is capable of hybridizing selectively to one or a subset of messenger RNAs transcribed from a given transcription unit of the sub genome, wherein at least one transcription unit of the sub genome encodes one or more messenger RNA splice variants.

35. The oligonucleotide library of claim 34, wherein said pathological tissue origin is cancer tissue.

36. An oligonucleotide library for detecting messenger RNAs that populate a sub transcriptome of a developmental stage, wherein the sub transcriptome comprises messenger RNAs transcribed from a multiplicity of transcription units that populate a sub genome of the developmental stage, wherein the library comprises a plurality of oligonucleotides, wherein each oligonucleotide in the plurality is capable of hybridizing selectively to one or a subset of messenger RNAs transcribed from a given transcription unit of the sub genome, wherein at least one transcription unit of the sub genome encodes one or more messenger RNA splice variants.

37. An oligonucleotide library for detecting messenger RNAs that populate a transcriptome of patients suffering from a disorder, wherein the transcriptome comprises messenger RNAs transcribed from a multiplicity of transcription units that populate a genome of patients suffering from the disorder, wherein the library comprises a plurality of oligonucleotides, wherein each oligonucleotide in the plurality is capable of hybridizing selectively to one or a subset of messenger RNAs transcribed from a given transcription unit of the genome, wherein at least one transcription unit of the genome encodes one or more messenger RNA splice variants.

38. The oligonucleotide library of claim 37, wherein said disorder is cancer.

39. A DNA microarray having spotted thereon a plurality of oligonucleotide sequences, wherein said plurality is provided by the oligonucleotide library of claim 29 or a subset thereof.

40. A DNA microarray having spotted thereon a plurality of oligonucleotide sequences, wherein said plurality is provided by the oligonucleotide library of claim 33 or a subset thereof.

41. A DNA microarray having spotted thereon a plurality of oligonucleotide sequences, wherein said plurality is provided by the oligonucleotide library of claim 34 or a subset thereof.

42. A DNA microarray having spotted thereon a plurality of oligonucleotide sequences, wherein said plurality is provided by the oligonucleotide library of claim 36 or a subset thereof.

43. A DNA microarray having spotted thereon a plurality of oligonucleotide sequences, wherein said plurality is provided by the oligonucleotide library of claim 37 or a subset thereof.

44. A method for expression profiling a cell or tissue sample that contains two or more RNAs of various abundances, comprising measuring hybridization signals of said sample to a plurality of oligonucleotide sequences, thereby determining the levels of said two or more RNAs in said sample and, where a RNA is transcribed from a transcription unit that has a set of splice variants, determining the total level of the set of splice variants, wherein said plurality is provided by the oligonucleotide library of claim 29 or a subset thereof.

45. The method of claim 44, wherein said hybridization signals are obtained from a nucleotide chip.

46. The method of claim 44, wherein said hybridization signals are obtained from an electrophoresis gel.

47. A method for expression profiling a cell or tissue sample that contains two or more RNAs of various abundances, comprising measuring hybridization signals of said sample to a plurality of oligonucleotide sequences, thereby determining the levels of said two or more RNAs in said sample and, where a RNA is transcribed from a transcription unit that has a set of splice variants, determining the total level of the set of splice variants, wherein said plurality is provided by the oligonucleotide library of claim 33 or a subset thereof.

48. The method of claim 47, wherein said hybridization signals are obtained from a nucleotide chip.

49. The method of claim 47, wherein said hybridization signals are obtained from an electrophoresis gel.

50. A method for expression profiling a cell or tissue sample that contains two or more RNAs of various abundances, comprising measuring hybridization signals of said sample to a plurality of oligonucleotide sequences, thereby determining the levels of said two or more RNAs in said sample and, where a RNA is transcribed from a transcription unit that has a set of splice variants, determining the total level of the set of splice variants, wherein said plurality is provided by the oligonucleotide library of claim 34 or a subset thereof.

51. The method of claim 50, wherein said hybridization signals are obtained from a nucleotide chip.

52. The method of claim 50, wherein said hybridization signals are obtained from an electrophoresis gel.

53. A method for expression profiling a cell or tissue sample that contains two or more RNAs of various abundances, comprising measuring hybridization signals of said sample to a plurality of oligonucleotide sequences, thereby determining the levels of said two or more RNAs in said sample and, where a RNA is transcribed from a transcription unit that has a set of splice variants, determining the total level of the set of splice variants, wherein said plurality is provided by the oligonucleotide library of claim 36 or a subset thereof.

54. The method of claim 53, wherein said hybridization signals are obtained from a nucleotide chip.

55. The method of claim 53, wherein said hybridization signals are obtained from an electrophoresis gel.

56. A method for expression profiling a cell or tissue sample that contains two or more RNAs of various abundances, comprising measuring hybridization signals of said sample to a plurality of oligonucleotide sequences, thereby determining the levels of said two or more RNAs in said sample and, where a RNA is transcribed from a transcription unit that has a set of splice variants, determining the total level of the set of splice variants, wherein said plurality is provided by the oligonucleotide library of claim 37 or a subset thereof.

57. The method of claim 56, wherein said hybridization signals are obtained from a nucleotide chip.

58. The method of claim 56, wherein said hybridization signals are obtained from an electrophoresis gel.

59. A double stranded RNA molecule based on an oligonucleotide selected from an oligonucleotide library for detecting messenger RNAs that populate a transcriptome, wherein the transcriptome comprises messenger RNAs transcribed from a multiplicity of transcription units that populate a genome, wherein the library comprises a plurality of oligonucleotides, wherein each oligonucleotide in the plurality is capable of hybridizing selectively to a set of messenger RNAs transcribed from a given transcription unit of the genome, wherein at least one transcription unit of the genome encodes one or more messenger RNA splice variants, wherein
the double-stranded RNA molecule comprises no more than 30 basepairs, wherein the double-stranded RNA molecule can interfere with translation of an mRNA.

60. An antisense molecule based on an oligonucleotide selected from an oligonucleotide library for detecting messenger RNAs that populate a transcriptome, wherein the transcriptome comprises messenger RNAs transcribed from a multiplicity of transcription units that populate a genome, wherein the library comprises a plurality of oligonucleotides, wherein each oligonucleotide in the plurality is capable of hybridizing selectively to a set of messenger RNAs transcribed from a given transcription unit of the genome, wherein at least one transcription unit of the genome encodes one or more messenger RNA splice variants, wherein
the antisense molecule comprises no more than 30 bases, wherein the double-stranded RNA molecule can interfere with translation of an mRNA.
